(19) United States
(12) Patent Application Publication
(10) Pub. No.: US 2025/0215379 A1
(43) Pub. Date: Jul. 3, 2025

(54) BIOMASS PREDICTIONS/ESTIMATION SYSTEM BASED ON PARAMETER SENSING FOR FIXED BED BIOREACTOR AND RELATED METHODS

(71) Applicant: UNIVERCELLS TECHNOLOGIES SA, Nivelles (BE)

(72) Inventors: Jean-Christophe Drugmand, Wavre (BE); Tania Pereira Chilima, Nivelles (BE)

(21) Appl. No.: 18/833,422

(22) PCT Filed: Jan. 26, 2023
(86) PCT No.: PCT/EP2023/051922

§ 371 Date: Jul. 26, 2024

Related U.S. Application Data
(60) Provisional application No. 63/325,701, filed on Mar. 31, 2022.
(60) Provisional application No. 63/303,133, filed on Jan. 26, 2022.

(51) Int. Cl. C12M 1/34; C12M 1/00; C12M 1/12; C12M 1/36

(52) U.S. Cl. CPC C12M 41/36; C12M 25/18; C12M 29/18; C12M 41/32; C12M 41/34; C12M 41/38; C12M 41/48

(57) ABSTRACT

A system for biomass sensing includes a fixed bed bioreactor including a container and a fixed bed disposed within such container. At least one sensor is for sensing one or more parameters representative of biomass in the fixed bed. A controller is adapted for correlating the one or more parameters to an amount of biomass of the fixed bed bioreactor, such as using a correlation model. Related systems and methods are also described.

This application claims the benefit of U.S. Provisional Patent Application Ser. Nos. 63/303,133, filed Jan. 26, 2022, and 63/325,701, filed Mar. 31, 2022, the disclosures of which are incorporated herein by reference.

### TECHNICAL FIELD

This document relates generally to a system using a correlation model for estimating or predicting an amount of biomass of a fixed bed bioreactor based on certain parameters thereof and related methods.

### BACKGROUND

Fixed bed bioreactors provide an optimal environment for growing biological cells (e.g., animal, insect or bacteria) and are able to achieve cell cultures having a high cell density, or “biomass.” Accurately determining the cell density during cell growth (or culturing) is a well-known challenge for users of fixed bed bioreactors. In particular, it is difficult to establish the number of cells immobilized on the fixed bed without directly accessing the bed. Such access to the fixed bed is often difficult and risks contamination of the bioreactor contents. Yet, the U.S. Food and Drug Administration's initiative of process analytical technology (PAT) also requires understanding the cell culturing process and timely monitoring of critical process parameters (CPP) that affect critical quality attributes (CQA), which necessitates such biomass determination for compliance.

Indirect techniques currently exist to monitor the density of animal cells inside a fixed bed bioreactor, but known examples of such techniques are complicated. For example, sampling a removable portion of the fixed bed with cells thereon may be accomplished to obtain an understanding of the cell density of the entire bioreactor. However, the cells may not be easily counted because they remain attached to the fixed bed portion obtained during the sampling. The cell count must be determined by lysing the cells and staining to count cell nuclei. The need to repeatedly perform this step during a cell culturing event or for each successive event increases the cost and complexity of the operation.

Frequent sampling of the fixed bed while maintaining aseptic conditions also presents challenges, since sampling typically requires accessing the fixed bed in the interior of the bioreactor, which may be sterile. Indirect measurement by capacitance biomass probes has been proposed, but is usually not accurate, precise, or repeatable. This is primarily due to the fact that only the volume surrounding the probe is measured. Insertion of the probe into the fixed bed is also not done consistently, which further contributes to these limitations.

Accordingly, a need is identified for a manner of determining the amount of biomass, or cell density, present in a fixed bed bioreactor. The technique would minimize or eliminate the potential risk of contamination associated with physical sampling of the fixed bed, while providing a more accurate indication of the fixed bed colonization than previously known techniques. This technique would allow for a real-time estimation of biomass production in the fixed bed, as well as potentially a prediction of future biomass level(s) based on current process conditions, thereby providing the operator with information needed to make real-time adjustments to achieve a desired outcome. The technique could also be applied via an automated system associated with the fixed bed bioreactor to dispense entirely with the need for operator intervention in order to estimate or predict the amount of biomass.

### SUMMARY

According to a first aspect of the disclosure, a biomass prediction/estimation system based on parameter sensing for a fixed bed bioreactor is provided. This system may include a bioreactor including a container (which may be sealed prior to or during use) and a fixed bed disposed within such container. At least one sensor is provided for sensing one or more parameters representative of biomass in the fixed bed. A controller is adapted to correlate the one or more parameters to an amount of biomass of the fixed bed.

In one embodiment, the one or more parameters representative of biomass comprise a cell culture byproduct, such as glucose or lactate. The at least one sensor may comprise a spectroscopic sensor, an enzymatic sensor, a gas sensor, or any combination thereof. The gas sensor and/or controller may be adapted to determine one or more of the following parameters: air and oxygen gas flow rate inputs, oxygen outlet concentration, oxygen transfer rate, oxygen uptake rate, carbon dioxide evolution rate, and respiratory quotient. The one or more parameters detected by sensor(s) may be selected from the group comprising glucose, lactate, Glu, Gln, Asp, Asn, NH3, or combinations thereof.

The sensor(s) may be associated with or connected directly to the bioreactor, or may be independent of it. For example, the system may employ an automated sampler to provide samples to the sensor(s). The sensor(s) may be associated with or positioned in a recirculation loop connected to the bioreactor, such as part of a dedicated line for drawing liquid from other than a surface of the bioreactor to minimize bubbles.

The controller may be adapted to estimate the amount of biomass in the bioreactor at a future time. The system (or controller) may further include a display for displaying the amount of biomass. The controller may be adapted to use a correlation model for receiving as an input the one or more parameters and processing the one or more parameters using the correlation model to output the amount of biomass of the fixed bed.

According to a further aspect of the disclosure, a system for biomass assessment includes a bioreactor including a container and a fixed bed disposed within such container. At least one sensor for sensing one or more parameters representative of biomass in the bioreactor. The at least one sensor is associated with a recirculation loop associated with the bioreactor.

Optionally, the system according to this aspect may include a controller adapted for correlating the one or more parameters to an amount of biomass of the bioreactor. The controller may be adapted to estimate the amount of biomass in the bioreactor at a future time. More specifically, the controller may be adapted to use a correlation model for receiving as an input the one or more parameters and processing (such as via a processor) the one or more parameters using the correlation model to output the amount of biomass of the fixed bed. A display may be associated with the controller for displaying the amount of biomass.

According to another aspect of the disclosure, a system for assessing biomass includes a bioreactor including a sealed container and a fixed bed disposed within such container. At least one sensor is provided for sensing one or more parameters representative of biomass in the fixed bed bioreactor. A controller is adapted to predict an amount of biomass in the fixed bed bioreactor at a future time based on the one or more parameters.

In one example, a display associated with the controller displays the amount of biomass that is predicted to be present in the bioreactor by the controller. To make the prediction, the controller may be adapted to use a correlation model for receiving as an input the one or more parameters and processing the one or more parameters using the correlation model to output the amount of biomass of the fixed bed.

In accordance with yet another aspect of the disclosure, a system comprises a bioreactor including a container and a fixed bed disposed within such container. At least one sensor is provided for sensing one or more parameters of a liquid provided to the at least one sensor by a conduit in fluid communication with the bioreactor. The conduit may comprise a dedicated line for drawing fluid from the bioreactor other than at a surface thereof to minimize the incidence of bubbles.

Optionally, the system may further include a controller adapted for correlating the one or more parameters to an amount of biomass of the bioreactor. The controller may be adapted to estimate the amount of biomass in the bioreactor at a future time. More specifically, the controller is adapted to use a correlation model for receiving as an input the one or more parameters and processing the one or more parameters using the correlation model to output the amount of biomass of the fixed bed. A display associated with the controller is provided for displaying the amount of biomass.

Yet another aspect of this disclosure pertains to a system for assessing biomass in a bioreactor including a container and a fixed bed disposed within such container and associated with a sensor for sensing one or more parameters representative of biomass in the fixed bed. The system comprises an automated sampler for obtaining a sample from the bioreactor and associating the sample with the sensor. The system further includes a controller adapted for correlating the one or more parameters obtained by and/or received from the sensor to an amount of biomass of the fixed bed.

The controller may be adapted to estimate the amount of biomass in the fixed bed at a future time. Specifically, the controller may use a correlation model for receiving as an input the one or more parameters and processing the one or more parameters using the correlation model to output the amount of biomass of the fixed bed. A display is associated with the controller for displaying the amount of biomass.

In other aspects, methods for assessing biomass include various steps, such as culturing cells in a fixed bed bioreactor, and during or after the culturing step, sensing, from cell culture fluid within or emanating from the bioreactor, one or more parameters representative of biomass in a fixed bed bioreactor (the one or more parameters including, for example, a metabolite level and/or a respiration level of a cell culture). The method may involve transmitting the one or more parameters to a controller and using the one or more parameters to estimate an amount of biomass of the fixed bed.

The using step may comprise using the controller and a correlation model to correlate the one or more parameters to the amount of biomass in the fixed bed. The method may involve manually inputting the one or more parameters into the controller. The amount of biomass may be a predicted future amount of biomass.

The sensing step may comprise providing the cell culture liquid to a metabolite sensor external to the fixed bed bioreactor. Alternatively or additionally, the sensing step comprises sensing the cell culture respiration level by monitoring one or more of air and oxygen gas flow rate inlets, oxygen outlet concentration, oxygen transfer rate, the oxygen uptake rate, carbon dioxide evolution rate, respiratory quotient, or any combination thereof.

Still a further method according to this disclosure pertains to determining biomass in a fixed bed bioreactor including cells, comprising measuring a parameter of the fixed bed bioreactor. The method further includes obtaining an actual measurement of a cell density for the fixed bed bioreactor. Still further, the method includes developing a correlation model for an estimated cell density based on the parameter and the actual measurement of cell density.

Using the correlation model, the method may involve estimating the cell density without the need for sampling the cells or directly measuring the cells, such as using an invasive probe. The estimating step may comprise further measuring the parameter using a sensor and applying the measured parameter to the correlation model. The actual measurement may comprises sampling the fixed bed bioreactor, or opening it to count at least a portion of the cells.

The method may further include the step of using the correlation model to provide an estimated cell density at a current time or at a future time. This information on estimated cell density may be used in determining when to infect or transfect the cells based on the estimated cell density.

A related aspect of the disclosure is a bioreactor system including a controller adapted to apply the correlation model obtained using the methods described herein to a measured parameter of a fixed bed bioreactor to estimate cell density, thus avoiding any need for sampling or measuring the cell density during cell culturing. A further related aspect of the disclosure is a bioreactor system including a controller adapted to apply the correlation model obtained using the methods described herein to predict cell density of a fixed bed bioreactor, thus avoiding any need for sampling or measuring the cell density during cell culturing.

A further aspect of the disclosure relates to a method for developing a final predictive model for cell density in a first fixed bed bioreactor. The method comprises developing a preliminary model correlating cell density with one or more parameters of one or more second fixed bed bioreactors. The method further comprises validating the preliminary model by obtaining an actual measurement of cell density from the one or more second fixed bed bioreactors to arrive at the final model. Still further, the method involves applying the final model to the first fixed bed bioreactor to estimate the cell density.

In one example, the step of developing the preliminary model comprises correlating cell density with metabolites in a plurality of second fixed bed bioreactors. Likewise, in this or another example, the obtaining step comprises obtaining one or more samples representative of cell density from the one or more second fixed bed bioreactors. The obtaining step may alternatively or additionally comprise opening the one or more second fixed bed bioreactors and counting at least a portion of the cells on a fixed bed therein.

### BRIEF DESCRIPTION OF THE DRAWING FIGURES

FIG. 1 schematically illustrates a biomass sensor system for a fixed bed bioreactor;

FIG. 2 schematically illustrates another embodiment of a biomass sensor system for a fixed bed bioreactor;

FIG. 3 schematically illustrates another embodiment of a biomass sensor system for a fixed bed bioreactor;

FIGS. 4, 4A, and 4B illustrate different locations of a metabolite sensor relative to input and output lines for the fixed bed bioreactor;

FIG. 5 illustrates a model correlating certain parameters with biomass, or cell density, in a fixed bed bioreactor;

FIGS. 6-7 graphically illustrate estimating and/or predicting biomass of the fixed bed bioreactor based on use of the correlation model and the corresponding metabolite data;

FIG. 8 is a flowchart showing one possible example of steps for developing and using the model; and

FIGS. 9 and 10 represent a graphical user interface for inputting and displaying current or updated information relating to the use of the model for correlating certain parameters with biomass in a fixed bed bioreactor.

### DETAILED DESCRIPTION

In one aspect, this disclosure pertains to a biomass estimation (current) or prediction (future) system for a fixed bed bioreactor. Specifically, the disclosed system may utilize real-time information relating to the cell culture (e.g., one or more parameters, such as respiratory information (oxygen consumption and carbon dioxide production) and/or metabolite information (glucose consumption and lactate production)) to obtain an accurate biomass estimation. Using this information, a correlation model may be employed to estimate cell density inside the fixed bed bioreactor. By employing this correlation model, the system may also be used to predict future conditions of the fixed bed in terms of biomass evolution (which could be an increase or decrease in cell density), and also allow for fully automated estimation and prediction to be achieved in a reliable, repeatable, lower risk, and cost effective manner, as compared to past approaches. The estimate (which may be a future prediction) may be used to assess when to take additional processing steps, such as for example steps to infect or transfect the cells.

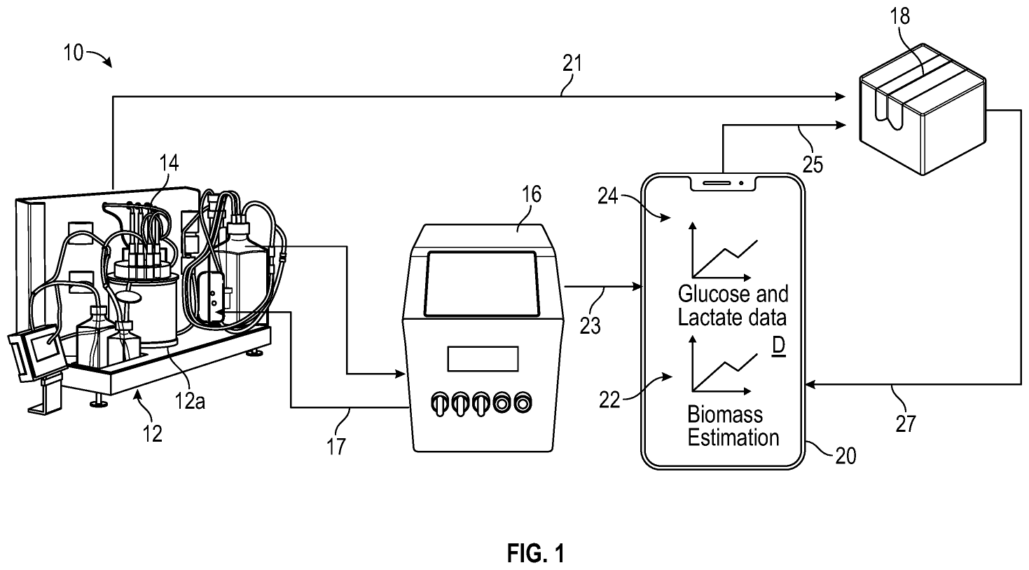

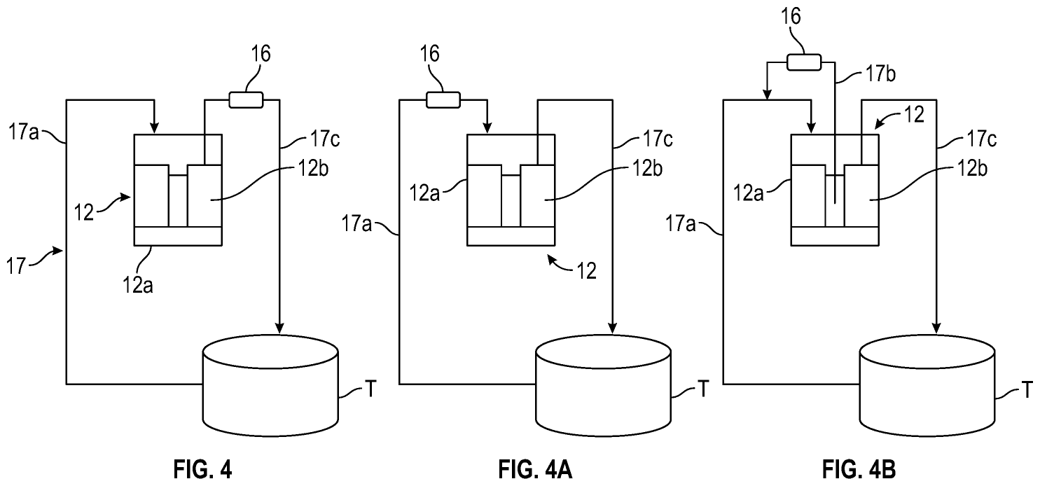

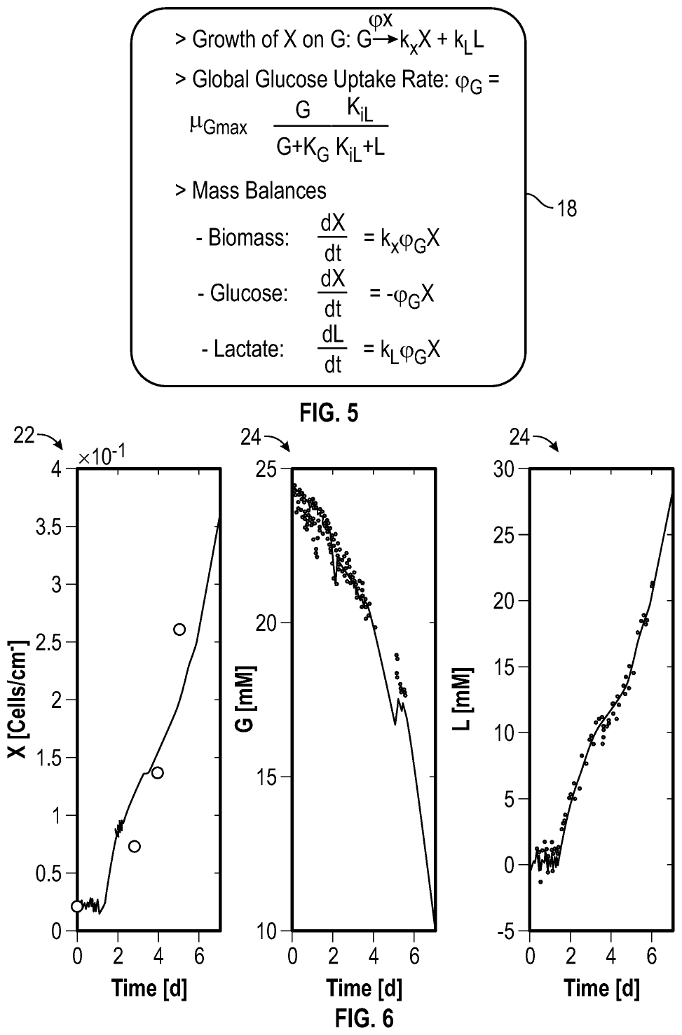

With reference to FIG. 1, an exemplary cell culturing system 10 includes a bioreactor 12, such as one including an internal structure for adherent or suspension cell growth. As shown in FIG. 1, as well as FIGS. 4, 4A, and 4B, the bioreactor 12 includes an outer vessel 12a or container including a fixed bed 12b as the internal structure for cell growth, which vessel 12a or bioreactor may be sealed to maintain an environment conducive to cell culturing (e.g., a sterile or aseptic environment). The fixed bed 12b may comprise, for example, an unstructured (packed) bed or a structured fixed bed. This bed 12b may comprise, for example, a 3-D printed matrix, or may be comprised of a woven or non-woven material(s) (such as, for example, one or more sheets of such a material in direct contact with each other or with interposed spacers, beads, hollow fibers, or any other suitable cell culture structure for promoting adherent cell growth). The bed 12b may be in any desired shape, orientation, or form, including for example 3D porous monoliths, stacked layers (see, e.g., U.S. Pat. No. 11,111,470, the disclosure of which is incorporated herein by reference), parallel layers arranged vertically, layers arranged in a spiral or wound configuration, or packed beds (see, e.g., U.S. Pat. No. 8,137,959, the disclosure of which is incorporated herein by reference).

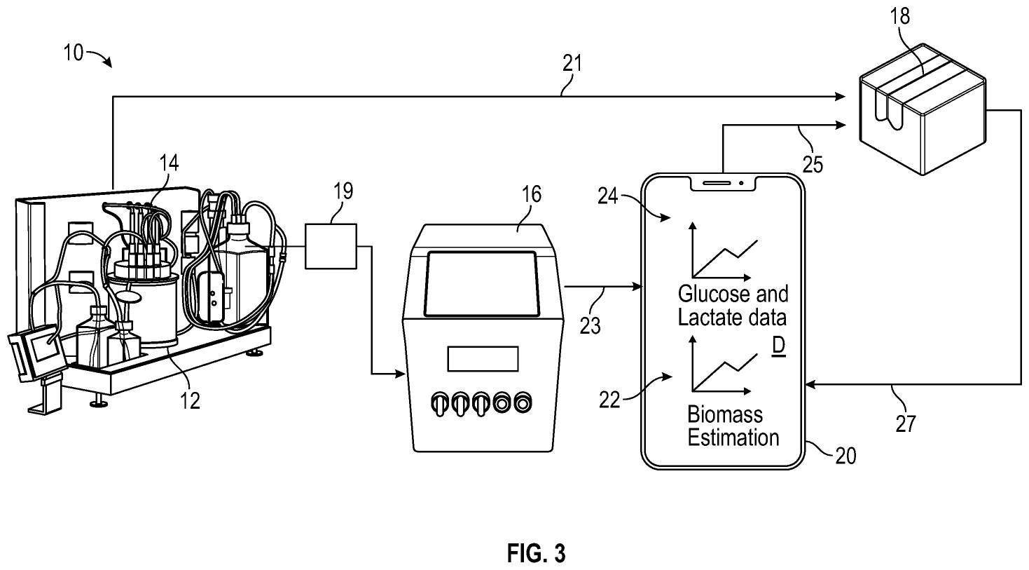

In one example, the system 10 involves having in-line or in-situ access to information for determining the biomass concentration of the fixed bed bioreactor 12. The information obtained from the fluid content within or emanating from the bioreactor may include information regarding one or more parameters of the bioreactor 12 capable of being determined in a non-invasive manner (e.g., through the use of in-line, in-situ sensors or analyzers, and possibly connected to an automated sampler 19, as shown in FIG. 3 and outlined further in the following description). Such parameters can include, for example, respiratory information and metabolite information. In particular, the respiratory information may involve one or more of the following parameters, as examples:
Real-time monitoring of air, oxygen (and potentially carbon dioxide) gas flow rate inlets (mass flow controllers).
Real-time monitoring of oxygen concentration in the culture media.
Real-time monitoring of the oxygen (and potentially carbon dioxide) outlet concentration (using a system such as the Bluesens analyzer).
Real-time calculation of the OTR (oxygen transfer rate) in the bioreactor.
Oxygen/CO₂ real-time oxygen mass balance calculation based on the oxygen uptake rate (OUR), carbon dioxide evolution rate (CDER) and respiratory quotient (RQ).

The information for such monitoring or calculation of such respiratory information may be obtained using one or more gas sensors 14 associated with the vessel, such as sealed container 12a, of the bioreactor 12. Such gas sensor(s) 14 may provide information in the form of output signals (arrow 21) to a controller including the model. The controller may comprise, for example, any microprocessor-based device, which may include an input device for receiving data, a microprocessor chip for processing the data, and an output device for transmitting processed data. This controller may include or be considered as a general purpose computer, special purpose computer, programmable logic controller, processor, microprocessor, or any other automated control unit able to computerize the calculations for applying the model 18 to estimate and/or predict the amount of biomass. This controller may thus form part of the system 10, as outlined in the following description, and may be a physical part of it or remote from it.

Collecting metabolite information from the bioreactor 12 may involve one or more of the following parameters, for example:
Real-time monitoring of the glucose and lactate concentration of the cell culture liquid;
Potentially monitoring other metabolites reflective of biomass, such as Glu, Gln, Asp, Asn, ammonia (NH3), pyruvate, etc.

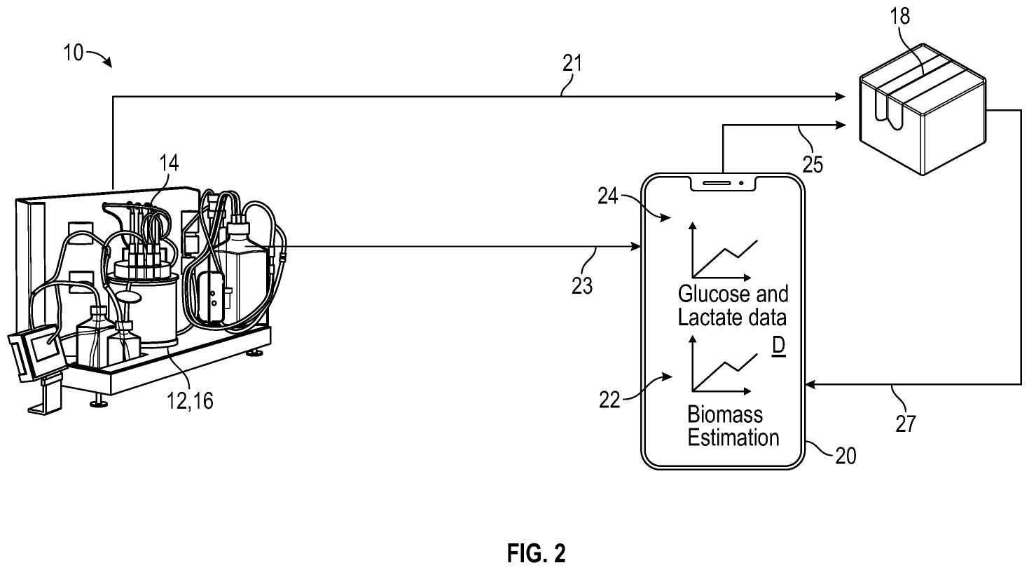

The information for monitoring one or more metabolite(s) may be obtained using one or more metabolite sensors 16 associated with the bioreactor 12, such as based on enzymatic technologies (such as a CCIT device) or spectroscopic technologies (such as an Irubis device). The arrangement for achieving sensing may comprise, for example: (1) an in-line sensor 16 connected to the bioreactor media recirculation loop 17 as shown in FIGS. 1 and 4; (2) an in-situ (i.e., directly integrated to the bioreactor) sensor 16, as shown in FIG. 2; and/or (3) as shown in FIG. 3, a sensor 16 associated with an automated sampler 19 (e.g., “Trace”/ISI/Novabiomedical) for sampling from the bioreactor 12.

In one particular example, the metabolite sensing function may involve the integration of a sensor into the system. For example, the metabolite sensor may comprise an in-line flow cell sensor 16 as shown in FIG. 1, capable of measuring metabolites flowing from the bioreactor 12 and providing measurements (such as in the form of output signals to the controller, such as computer 20 or other processor) representative of the cell culture conditions inside the associated fixed bed 12b. One way of integrating such a sensor 16 into a system 10 including a fixed bed bioreactor 12 is to position it “in line” with a conduit for delivering fluid (supernatant) from this bioreactor in real time, such as by way of the recirculation loop 17 (with suitable filtering if necessary).

As indicated in FIGS. 4, 4A, and 4B, which depict the recirculation loop 17 in communication with the fixed bed bioreactor 12 and a recirculation tank T, the sensor 16 may be arranged in an input line 17a (FIG. 4A) or a dedicated line 17b in communication between the bioreactor 12 and the input line 17a (FIG. 4B). This is contrasted with an output line 17c (FIG. 4), which may include bubbles in the liquid obtained from the bioreactor 12, which bubbles could impact the metabolite sensing in some instances (such as if the sensor is sensitive to the presence of bubbles). The dedicated line 17b may comprise, for example, a conduit positioned below a surface of the liquid in the bioreactor 12, as shown in FIG. 4B.

As noted previously, one or more different types of metabolite sensor(s) 16 could be used in connection with the system 10. For example, one or more spectroscopic (e.g., Irubis) sensors could be provided in the recirculation loop 17. One or more enzymatic (e.g., CCIT) sensors may also be used, alone or in combination with other sensors. As noted above, in situations where any of the sensor(s) 16 used may be sensitive to bubbles in terms of producing accurate measurements, a “bubble free” set up may be used.

Using information from one or both of the gas sensor(s) 14 or metabolite sensor(s) 16, biomass estimation may be automatically calculated in-real time using a correlation model 18. The model 18 may be implemented by a controller, such as for example a computer 20 as shown schematically, also forming part of the system 10. As further illustrated schematically in FIGS. 1-3, the computer 20 receives as an input 23 metabolite information representative of parameters from the sensor(s) 14, 16, applies such information to the model 18 via processor (or which information may be manually inputted into the model), as indicated by arrow 25, and by such processing capability generates an output 27 from the model 18 of an estimated or predicted biomass amount (e.g., cell density) in the fixed bed bioreactor 12.

The nature of the model(s) 18 used may vary, versions of which are known to skilled artisans. One example of a cell growth kinetic correlation model 18 based on metabolites is provided in FIG. 4, which is an example of an unstructured mechanistic kinetic model with macroscopic mass balance and Monod type equation with inhibitor factors. The selected model 18 may also include a function for calculating the predicted state estimates, the corrected state estimates, the corresponding gains used to calculate these estimates, the associated prediction and estimation error covariances corresponding to these estimates, and the estimated output, such as a Discrete-Discrete Extended Kalman Filter, such as per the following example:
1. Prediction equations in discrete time (recurrence equations directly computing solutions from one simulation time point tₛ to the next one tₛ₊₁) with sampling time Δtₛ=tₛ−tₛ₋₁:

### Prediction Equations

$${x}_{k}^{-} = f ( {x}_{k - 1} , ϑ )$$

Pₖ⁻=Aₖ₋₁Pₖ₋₁Aₖ₋₁^{T}+Qₓ

where
P represents the covariance matrix of the state estimation errors

$$A = \frac{\partial f ( x , ϑ )}{\partial x} = [ \begin{matrix}\frac{\partial {f}_{X}}{\partial X} & \frac{\partial {f}_{X}}{\partial G} & \frac{\partial {f}_{X}}{\partial L} \\ \frac{\partial {f}_{G}}{\partial X} & \frac{\partial {f}_{G}}{\partial G} & \frac{\partial {f}_{G}}{\partial L} \\ \frac{\partial {f}_{L}}{\partial X} & \frac{\partial {f}_{L}}{\partial G} & \frac{\partial {f}_{L}}{\partial L}\end{matrix} ]$$

$${P}_{0} = [ \begin{matrix}{P}_{X} ( 0 ) & 0 & 0 \\ 0 & {P}_{G} ( 0 ) & 0 \\ 0 & 0 & {P}_{L} ( 0 )\end{matrix} ] and {Q}_{x} = [ \begin{matrix}{Q}_{x X} & 0 & 0 \\ 0 & {Q}_{xG} & 0 \\ 0 & 0 & {Q}_{x L}\end{matrix} ] :$$

tuning parameters

### Details for Computing Aₖ₋₁

Time index (k or k−1) not mentioned for the sake of simplicity.

$$A = \frac{\partial f ( x , ϑ )}{\partial x} = [ \begin{matrix}\frac{\partial {f}_{X}}{\partial X} & \frac{\partial {f}_{X}}{\partial G} & \frac{\partial {f}_{X}}{\partial L} \\ \frac{\partial {f}_{G}}{\partial X} & \frac{\partial {f}_{G}}{\partial G} & \frac{\partial {f}_{G}}{\partial L} \\ \frac{\partial {f}_{L}}{\partial X} & \frac{\partial {f}_{L}}{\partial G} & \frac{\partial {f}_{L}}{\partial L}\end{matrix} ] \begin{matrix}{f}_{X} = X + Δ {tk}_{X} {φ}_{G} ( G , L ) X \\ {f}_{G} = G - Δ t {φ}_{G} ( G , L ) X \\ {f}_{L} = L + Δ {tk}_{L} {φ}_{G} ( G , L ) X\end{matrix}$$

$$\begin{matrix}\frac{\partial {f}_{X}}{\partial X} = 1 + Δ {tk}_{X} {φ}_{G} \\ \frac{\partial {f}_{X}}{\partial G} = Δ {tk}_{X} \frac{\partial {φ}_{k}}{\partial G} X \\ \frac{\partial {f}_{X}}{\partial L} Δ {tk}_{X} \frac{\partial {φ}_{G}}{\partial L} X\end{matrix} \begin{matrix}\frac{\partial {f}_{G}}{\partial X} = - Δ t {φ}_{G} \\ \frac{\partial {f}_{G}}{\partial G} = 1 - Δ t \frac{\partial {φ}_{G}}{\partial G} X \\ \frac{\partial {f}_{G}}{\partial L} = - Δ t \frac{\partial {φ}_{G}}{\partial L} X\end{matrix} \begin{matrix}\frac{\partial {f}_{L}}{\partial X} = Δ {tk}_{L} {φ}_{G} \\ \frac{\partial {f}_{L}}{\partial G} = Δ {tk}_{X} \frac{\partial {φ}_{G}}{\partial G} X \\ \frac{\partial {f}_{L}}{\partial L} = 1 + Δ {tk}_{L} \frac{\partial {φ}_{G}}{\partial L} X\end{matrix}$$

$${φ}_{G} = {μ}_{Gmax} \frac{G}{G + {K}_{G}} \frac{{K}_{iL}}{{K}_{iL} + L} \frac{\partial {φ}_{G}}{\partial G} = {μ}_{Gmax} \frac{{K}_{G}}{{( G + {K}_{G} )}^{2}} \frac{{K}_{iL}}{{K}_{iL} + L}$$

$$\frac{\partial {φ}_{G}}{\partial L} = - {μ}_{Gmax} \frac{G}{G + {K}_{G}} \frac{{K}_{iL}}{{( {K}_{iL} + L )}^{2}}$$

2. Correction equations in discrete time (at each new measurement time point tₖ) within a sampling time Δtₖ.

### Correction Equations (at Each New Measurement Time Point Tₖ)

$${K}_{k} = {P}_{k}^{-} {{C}^{T} ( {CP}_{k}^{-} {C}^{T} + {Q}_{y k} )}^{- 1}$$

Kₖ∈ [symbol] : correction gain at time tₖ
Pₖ⁻: predicted value of P at time tₖ (see prediction equations)

$${Q}_{y k} = [ \begin{matrix}{Q}_{G} ( {t}_{k} ) & 0 \\ 0 & {Q}_{L} ( {t}_{k} )\end{matrix} ] :$$

variances of the measurement errors on G and L at time tₖ

$${x}_{k} = {x}_{k}^{-} + {K}_{k} ( {y}_{k} - {Cx}_{k}^{-} )$$

xₖ⁻: predicted values of x at time tₖ (see prediction equations)

$${P}_{k} = {P}_{k}^{-} - {K}_{k} C {P}_{k}^{-}$$

After correction: use of the prediction equations for predicting xₖ₊₁⁻ and Pₖ₊₁⁻ from the corrected values xₖ and Pₖ

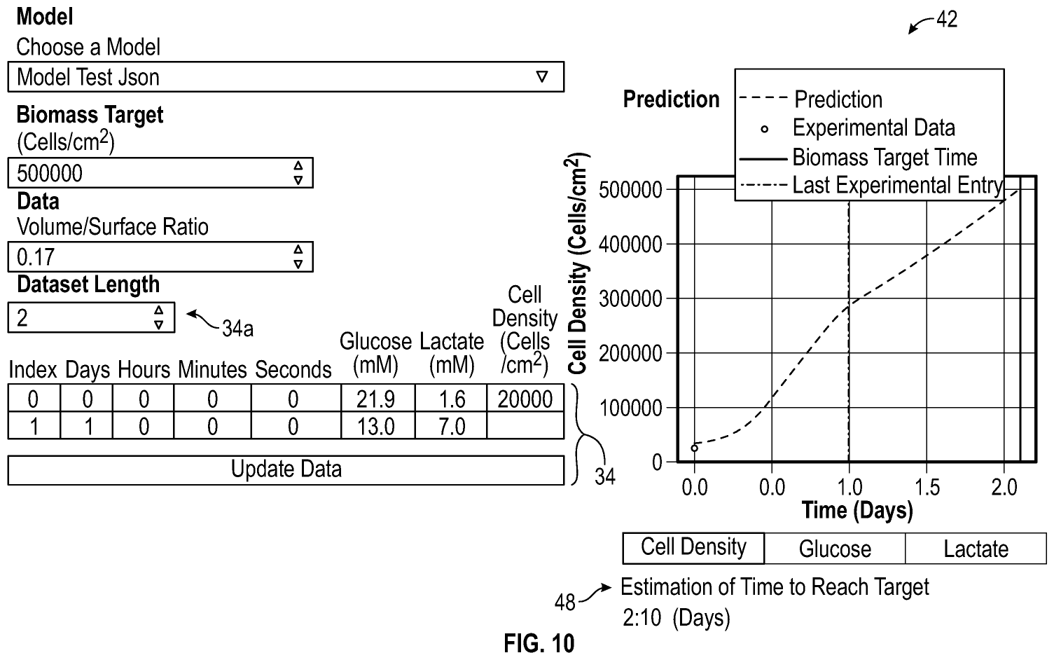

Using the selected correlation model 18, the computer 20 may correlate the information on one or more parameters, such as oxygen consumption and metabolites, to provide a biomass estimation 22. As indicated in FIGS. 1-3, this estimation 22 may comprise an estimated biomass amount associated with the fixed bed bioreactor 12, and may be displayed graphically to the user in an associated display D (see also FIGS. 9-10) forming part of the system 10. This display D may be part of or associated with a computer 20 serving as the controller, as shown in FIGS. 1-3. The display D may also provide the measured parameter levels 24 (e.g., one or more metabolites), which are shown in more detail in FIG. 6. Thus, an indication of biomass is provided without the need for sampling or other invasive techniques, and aseptic conditions may be reliably maintained.

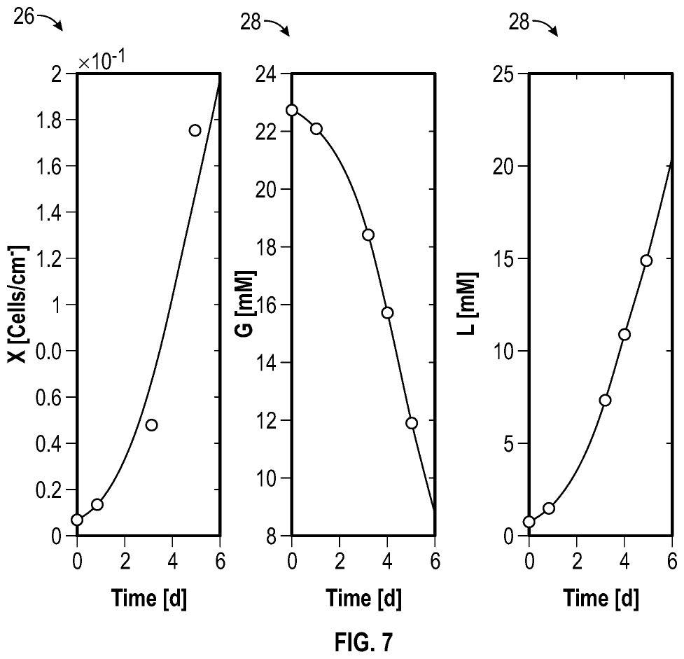

Using predictive techniques (e.g., an extended Kalman filter, such as mentioned above), the model 18 may also be used to predict a future amount of biomass in the fixed bed bioreactor 12, as shown by graph 26 in FIG. 7. Predictions may also be made regarding the parameter (e.g., metabolite) levels, as shown by graphs 28 (glucose and lactate, as examples). This information may be displayed via computer 20, either in graphical or numerical form, for a given point in time or over a range of times. This information allows the operator to understand the future biomass generation potential of the fixed bed bioreactor 12 (whether positive or negative in amount), without directly accessing the fixed bed or otherwise sampling it to obtain a direct measurement of cell density. This not only avoids the concerns over breaching sterility, but also the limitations noted above with respect to past approaches for in-situ sensing of biomass, such as cell density probes.

Sensing the parameters of the bioreactor 12 to determine existing or predict future biomass levels in the fixed bed may be done periodically, as determined to be necessary for a particular situation. Whether done in an automated fashion or manually, the sampling and/or sensing may be done with a high frequency, such as for example every few seconds. Depending on the circumstances, it may also be done with a lower frequency, such as once an hour, once per day, or perhaps longer,

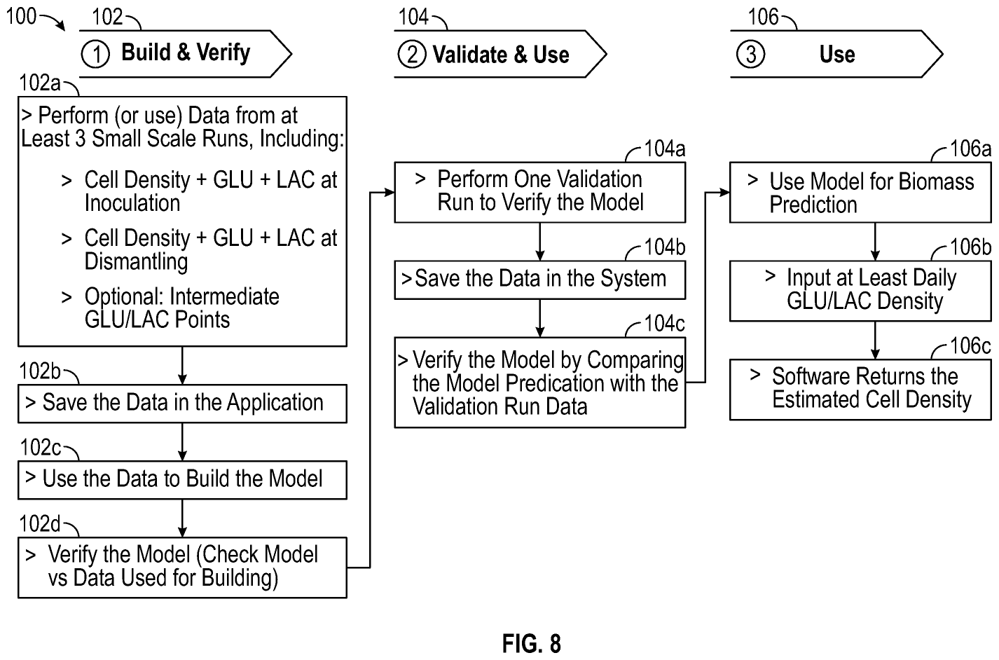

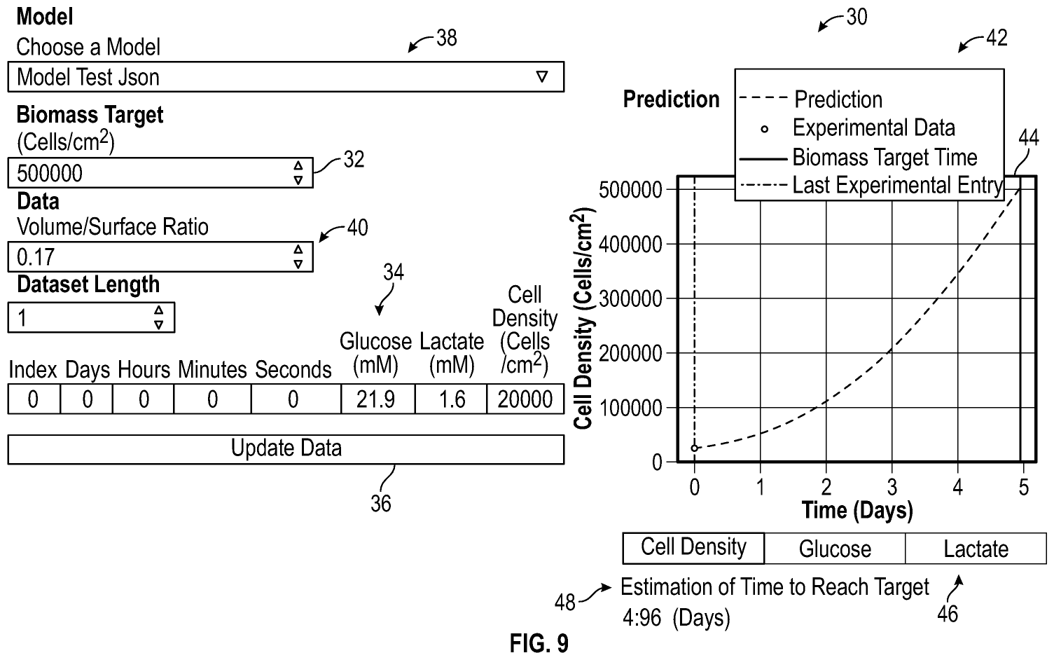

According to a further aspect of the disclosure, development of a custom correlation model in connection with sampling one or more test runs of the bioreactor may be done in order to allow for later, real-time non-invasive modeling of the biomass production. As indicated in the flow chart provided in FIG. 8, this may be achieved by employing a biomass/metabolite correlation method 100, which is independent of the type of cells being cultured. This method 100 may involve conducting a plurality of calibration runs of a fixed bed bioreactor, potentially at different scales, in order to develop a preliminary correlation model, as indicated at step 102. This step 102 may involve periodically measuring or sensing one or more parameters indicative of biomass production (such as metabolites), combined with actual measurement of the amount of biomass associated with the fixed bed (either during bioprocessing via sampling, or once completed).

Once full parameter estimation and validation of the preliminary model is completed (step 104), this model may then be used as a final model in connection with a fixed bed bioreactor without the need for sampling (step 106), in order to provide a real-time indication of biomass production without the risk of contamination. More specifically, the process of creating and validating the preliminary correlation model may include conducting a plurality of runs of a small scale version of the fixed bed bioreactor. As shown in FIG. 8 at sub-step 102a, this may involve measuring one or more parameters, such as glucose and lactate production, while concurrently assessing the biomass production of the fixed bed bioreactor in order to establish corresponding cell density values. This may be done in a variety of ways, including by dismantling the bioreactor once bioprocessing is completed, or by sampling during the bioprocessing event. A particular example of a suitable sampling system for a fixed bed bioreactor may be found in US2021/009933, the disclosure of which is incorporated herein by reference.

In any case, using the measurements obtained and saved (sub-step 102b), the preliminary model correlating the one or more parameters with biomass may be developed, as indicated by sub-step 102c. This could also be accomplished, for example, using a relatively small scale version of the fixed bed bioreactor, such as one designed to be easily opened at the end of the culture, in order to facilitate the sampling of the fixed-bed material for the cell density estimation after cell lysis and staining to count cell nuclei. Such an easy access bioreactor may have a lid removably secured to the bioreactor vessel.

Optionally, a plurality of mid-scale confirmation runs may be conducted. This may be done to confirm the scalability of the developed model, and fine-tune the parameters of this model. This may also involve measuring the same parameters as per the initial “small scale” step, combined with sampling, as previously noted.

Verification of the preliminary model may also be performed as part of the building step 102. This may involve, for instance, checking the model versus the data used to build it, as indicated at sub-step 102d.

The validation step 104 may also comprise a sub-step 104a of performing a validation run to verify the preliminary model. The data may be saved, as indicated by sub-step 104b. A verification sub-step 104c may also be performed by comparing the prediction obtained by the model with the data from the validation run at step 104a.

Once the preliminary model is fully developed using these steps, it may be applied as a final correlation model to larger scale runs of a fixed bed bioreactor 12 for biomass prediction (sub-step 106a) without the need for sampling. This may involve, for example, inputting information regarding the metabolite values (e.g. daily glucose/lactate measurements), which may be done manually or automatically (sub-step 106b). The final model may then output the estimated cell density for such condition(s) (sub-step 106c).

Accordingly, as can be appreciated, the disclosed biomass prediction/estimation techniques may be used to provide a real-time estimation of the cell density based on the measurement of the metabolites and/or respiration levels, independent of the type of cells and depending on the process parameters. As noted above and further in the following description, future predictions of the cell density may also be made to forecast the productivity of the fixed bed bioreactor, including with the step of measuring the initial cell density in the inoculum in order to perform the estimation. This information may then be used to determine when to conduct further process steps, such as infection or transfection, or whether to adjust other aspects or parameters of the bioprocessing operation to achieve a particular outcome in terms of considerations like biomass, time, or others.

As noted above and shown schematically in FIG. 1, the biomass estimation/prediction model 18 may be operated as an algorithm or program on a computer 20. The computer 20 may be programmed to display a graphical user interface 30, as shown in FIGS. 9-10. This graphical user interface 30 may include an input 32 for inputting a biomass target, as well as for an indication 34 of the measured metabolite(s) (which may be manually entered or automatically obtained via the above-mentioned sensors). The interface may also include an activation button 36 to update the model and display, or this could be done automatically. A selector 38 may also be provided for selecting from among available biomass estimation or prediction models, if applicable. The interface 30 may further allow for additional information to be inputted as necessary (e.g., a further parameter of the bioreactor and associated fixed bed, such as the volume-to-surface ratio 40).

The interface 30 may further provide an output 42 from the model 18 regarding the prediction of the time to reach the target, such as in the form of a graphical representation 44. The output 42 may, for example, indicate the estimated or predicted amount of biomass. The interface 30 may provide for the selected display of the corresponding level of one or more metabolites, such as by way of corresponding selection buttons 46 to toggle between the information displayed. A numerical calculation 48 of the estimated time to reach the particular target may also be displayed in addition to the graphical representation 44.

As indicated in FIG. 10, the metabolite values may be updated over time (again, manually or automatically), which may be shown in indication 34 in plural lines, which may involve using an input 34a to increase the length of the data set. In such case, the output 42 may be revised to indicate the biomass level at that particular time, and the graphical representation 44 updated accordingly to facilitate the user's understanding of the situation in real time. The output 42 may also revise the estimated numerical time calculation 48 to achieve the desired biomass target using the updated information obtained.

While respiration, glucose, and lactate are mentioned as possible parameters that may be sensed and correlate to cell density, other parameters may also be used. For example, the parameters may related to the consumption of nutrients, such as glutamine, pyruvate, asparagine and generally speaking all the amino acids, intermediate of the Krebs cycle and sugars (C5 and C6) present into the culture media. The parameters may also relate to the production of by-products, such as ammonia, ethanol, Alanine, etc. Additional process parameters that could be used in the model include pH, temperature, and stirring speed of the bioreactor (as this could impact the oxygen transfer rate). Parameters concerning the feeding strategy could also being used in connection with the model, such as the amount of media (ml/cm²), flow (perfusion/recirculation) rate of the media inlet, media exchanges, etc.

Summarizing the various aspects to which this disclosure may pertain, the following items are identified, which may be arranged in any combination:
1. A system for assessing biomass for a bioreactor including a container and a fixed bed disposed within such container, comprising:
   at least one sensor for sensing one or more parameters representative of biomass in the fixed bed; and/or
   a controller adapted to correlate the one or more parameters to an amount of biomass of the fixed bed.
2. The system of item 1, wherein the one or more parameters representative of biomass comprise a cell culture byproduct, such as glucose or lactate.
3. The system of item 1 or item 2, wherein the at least one sensor comprises a spectroscopic sensor.
4. The system of any of items 1-3, wherein the at least one sensor comprises an enzymatic sensor.
5. The system of any of items 1-4, wherein the at least one sensor comprises a gas sensor associated with the bioreactor.
6. The system of item 5, wherein the gas sensor and/or controller are adapted to determine one or more of air and oxygen gas flow rate inputs, oxygen outlet concentration, oxygen transfer rate, oxygen uptake rate, carbon dioxide evolution rate, and respiratory quotient.
7. The system of any of items 1-6, wherein the at least one sensor is positioned in the bioreactor, or in a recirculation loop connected to the bioreactor, such as part of a dedicated line for drawing liquid from other than a surface of the bioreactor to minimize bubbles.
8. The system of any of items 1-7, wherein the at least one sensor is associated with an automated sampler for sampling from the bioreactor.
9. The system of any of items 1-8, wherein the controller is adapted to estimate the amount of biomass in the bioreactor at a future time.
10. The system of any of items 1-9, further including a display associated with the controller for displaying the amount of biomass.
11. The system of any of items 1-10, wherein the one or more parameters are selected from the group comprising glucose, lactate, Glu, Gln, Asp, Asn, NH3, or combinations thereof.
12. The system of any of items 1-11, wherein the controller is adapted to use a correlation model for receiving as an input the one or more parameters and processing the one or more parameters using the correlation model to output the amount of biomass of the fixed bed.
13. The system of any of items 1-12, wherein the bioreactor comprises a sealed container.
14. A system for assessing biomass for a bioreactor including a container and a fixed bed disposed within such container and including a recirculation loop, comprising at least one sensor for sensing one or more parameters representative of biomass in the bioreactor, the at least one sensor associated with the recirculation loop.
15. The system of item 14, further including a controller adapted for correlating the one or more parameters to an amount of biomass of the bioreactor.
16. The system of item 15, wherein the controller is adapted to estimate the amount of biomass in the bioreactor at a future time.
17. The system of item 14 or item 15, wherein the controller is adapted to use a correlation model for receiving as an input the one or more parameters and processing the one or more parameters using the correlation model to output the amount of biomass of the fixed bed.
18. The system of any of items 15-17, further including a display associated with the controller for displaying the amount of biomass.
19. A system for assessing biomass for a bioreactor including a container and a fixed bed disposed within such container, comprising:
   at least one sensor for sensing one or more parameters representative of biomass in the fixed bed bioreactor; and/or
   a controller adapted to predict an amount of biomass in the fixed bed bioreactor at a future time based on the one or more parameters.
20. The system of item 19, further including a display associated with the controller for displaying the amount of biomass.
21. The system of item 19 or item 20, wherein the controller is adapted to use a correlation model for receiving as an input the one or more parameters and processing the one or more parameters using the correlation model to output the amount of biomass of the fixed bed.
22. A system, comprising:
   a bioreactor including a container and a fixed bed disposed within such container; and/or
   at least one sensor for sensing one or more parameters of a liquid provided to the at least one sensor by a conduit in fluid communication with the bioreactor, and which conduit may comprise a dedicated line for drawing fluid from the bioreactor other than at a surface thereof to minimize the incidence of bubbles.
23. The system of item 22, further including a controller adapted for correlating the one or more parameters to an amount of biomass of the bioreactor.
24. The system of item 23, wherein the controller is adapted to estimate the amount of biomass in the bioreactor at a future time.
25. The system of item 23 or item 24, wherein the controller is adapted to use a correlation model for receiving as an input the one or more parameters and processing the one or more parameters using the correlation model to output the amount of biomass of the fixed bed.
26. The system of any of items 23-25, further including a display associated with the controller for displaying the amount of biomass.
27. A system for assessing biomass in a bioreactor including a container and a fixed bed disposed within such container and associated with a sensor for sensing one or more parameters representative of biomass in the fixed bed, comprising:
   an automated sampler for obtaining a sample from the bioreactor and associating the sample with the sensor; and/or
   a controller adapted for correlating the one or more parameters from the sensor to an amount of biomass of the fixed bed.
28. The system of item 27, wherein the controller is adapted to estimate the amount of biomass in the fixed bed at a future time.
29. The system of item 27 or item 28, wherein the controller is adapted to use a correlation model for receiving as an input the one or more parameters and processing the one or more parameters using the correlation model to output the amount of biomass of the fixed bed.
30. The system of any of items 27-29, further including a display associated with the controller for displaying the amount of biomass.
31. A method for biomass assessing, comprising:
   culturing cells in a fixed bed bioreactor; and/or
   during or after the culturing step, sensing, from cell culture fluid within or emanating from the bioreactor, one or more parameters representative of biomass in a fixed bed bioreactor, the one or more parameters including a metabolite level and/or a respiration level of a cell culture; and/or
   transmitting the one or more parameters to a controller; and/or
   using the one or more parameters to estimate an amount of biomass of the fixed bed.
32. The method of item 31, wherein the using step comprises using the processor and a correlation model to correlate the one or more parameters to the amount of biomass in the fixed bed.
33. The method of item 31 or item 32, further including the step of manually inputting the one or more parameters into the controller.
34. The method of any of items 31-33, wherein the amount of biomass is a predicted future amount of biomass.
35. The method of any of items 31-34, wherein the sensing step comprises providing the cell culture liquid to a metabolite sensor external to the fixed bed bioreactor.
36. The method of any of items 31-35, wherein the sensing step comprises sensing the cell culture respiration level by monitoring one or more of air and oxygen gas flow rate inlets, oxygen outlet concentration, oxygen transfer rate, the oxygen uptake rate, carbon dioxide evolution rate and respiratory quotient.
37. A method for assessing biomass in a fixed bed bioreactor including cells, comprising:
   measuring a parameter of the fixed bed bioreactor; and/or
   obtaining an actual measurement of a cell density for the fixed bed bioreactor; and/or
   developing a correlation model for an estimated cell density based on the parameter and the actual measurement of cell density.
38. The method of item 37, further including the step of estimating the cell density using the correlation model without the need for sampling.
39. The method of item 37, wherein the estimating step comprises further measuring the parameter using a sensor and applying the measured parameter to the correlation model.
40. The method of any of items 37-39, wherein the step of obtaining the actual measurement comprises sampling the fixed bed bioreactor.
41. The method of any of items 37-40, further including the step of using the correlation model to provide an estimated cell density at a current time or at a future time.
42. The method of any of items 31-41, further including the step of determining when to infect or transfect the cells based on the estimated cell density.
43. A bioreactor system including a controller adapted to apply the correlation model obtained using the method of any of items 37-42 to a measured parameter of a fixed bed bioreactor to estimate cell density, thus avoiding any need for sampling or measuring the cell density during cell culturing.
44. A bioreactor system including a controller adapted to apply the correlation model obtained using the method of items 37-42 to predict cell density of a fixed bed bioreactor, thus avoiding any need for sampling or measuring the cell density during cell culturing.
45. A method for developing a final predictive model for cell density in a first fixed bed bioreactor, comprising:
   developing a preliminary model correlating cell density with one or more parameters of one or more second fixed bed bioreactors; and/or
   validating the preliminary model by obtaining an actual measurement of cell density from the one or more second fixed bed bioreactors to arrive at the final model; and/or
   applying the final model to the first fixed bed bioreactor to estimate the cell density.
46. The method of item 45, wherein the step of developing the preliminary model comprises correlating cell density with metabolites in a plurality of second fixed bed bioreactors.
47. The method of item 45 or item 46, wherein the obtaining step comprises obtaining one or more samples representative of cell density from the one or more second fixed bed bioreactors.
48. The method of any of items 45-48, wherein the obtaining step comprises opening the one or more second fixed bed bioreactors and counting at least a portion of the cells on a fixed bed therein.

For purposes of this disclosure, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

While certain embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. A system for assessing biomass for a bioreactor including a container and a fixed bed disposed within such container, comprising:
at least one sensor for sensing one or more parameters representative of biomass in the fixed bed; and
a controller adapted to correlate the one or more parameters to an amount of biomass of the fixed bed;
wherein the one or more parameters representative of biomass comprise a cell culture byproduct.

2. The system of claim 1, wherein the one or more parameters representative of biomass comprise glucose or lactate as the cell culture byproduct.

3. The system of claim 1, wherein the at least one sensor comprises a spectroscopic sensor.

4. The system of claim 1, wherein the at least one sensor comprises an enzymatic sensor.

5. The system of claim 1, wherein the at least one sensor comprises a gas sensor associated with the bioreactor.

6. The system of claim 5, wherein the gas sensor and/or controller are adapted to determine one or more of air and oxygen gas flow rate inputs, oxygen outlet concentration, oxygen transfer rate, oxygen uptake rate, carbon dioxide evolution rate, and respiratory quotient.

7. The system of claim 1, wherein the at least one sensor is positioned in the bioreactor, or in a recirculation loop connected to the bioreactor, such as part of a dedicated line for drawing liquid from other than a surface of the bioreactor to minimize bubbles.

8. The system of claim 1, wherein the at least one sensor is associated with an automated sampler for sampling from the bioreactor.

9. The system of claim 1, wherein the controller is adapted to estimate the amount of biomass in the bioreactor at a future time.

10. The system of claim 1, further including a display associated with the controller for displaying the amount of biomass.

11. The system of claim 1, wherein the one or more parameters are selected from the group comprising glucose, lactate, Glu, Gin, Asp, Asn, NH3, or combinations thereof.

12. The system of claim 1, wherein the controller is adapted to use a correlation model for receiving as an input the one or more parameters and processing the one or more parameters using the correlation model to output the amount of biomass of the fixed bed.

13. The system of claim 1, wherein the bioreactor comprises a sealed container.

14. A system for assessing biomass, comprising:
a bioreactor including a container and a fixed bed disposed within such container, the bioreactor associated with a recirculation loop; and
at least one sensor for sensing one or more parameters representative of biomass in the bioreactor, the at least one sensor associated with the recirculation loop.

15. The system of claim 14, further including a controller adapted for correlating the one or more parameters to an amount of biomass of the bioreactor.

16. The system of claim 15, wherein the controller is adapted to estimate the amount of biomass in the bioreactor at a future time.

17. The system of claim 15, wherein the controller is adapted to use a correlation model for receiving as an input the one or more parameters and processing the one or more parameters using the correlation model to output the amount of biomass of the fixed bed.

18. The system of claim 15, further including a display associated with the controller for displaying the amount of biomass.

19.-21. (canceled)

22. A system, comprising:
a bioreactor including a container and a fixed bed disposed within such container; and
at least one sensor for sensing one or more parameters of a liquid provided to the at least one sensor by a conduit in fluid communication with the bioreactor, and which conduit comprises a dedicated line for drawing fluid from the bioreactor other than at a surface thereof to minimize the incidence of bubbles.

23. The system of claim 22, further including a controller adapted for correlating the one or more parameters to an amount of biomass of the bioreactor.

24. The system of claim 23, wherein the controller is adapted to estimate the amount of biomass in the bioreactor at a future time.

25. The system of claim 23, wherein the controller is adapted to use a correlation model for receiving as an input the one or more parameters and processing the one or more parameters using the correlation model to output the amount of biomass of the fixed bed.

26. The system of claim 23, further including a display associated with the controller for displaying the amount of biomass.

27.-48. (canceled)
